(19) United States
(12) Patent Application Publication
(10) Pub. No.: US 2025/0157634 A1
(43) Pub. Date: May 15, 2025

(54) CARE MANAGEMENT SUPPORT METHOD, INFORMATION PROCESSING APPARATUS, SYSTEM, AND PROGRAM

(71) Applicant: Sompo Care Inc., Shinagawa-ku, Tokyo (JP)

(72) Inventors: Yohei MIYAGATA, Shinjuku-ku, Tokyo (JP); Keiko HORI, Shinjuku-ku, Tokyo (JP); Masaki TORII, Shinagawa-ku, Tokyo (JP)

(73) Assignee: Sompo Care Inc., Shinagawa-ku, Tokyo (JP)

(21) Appl. No.: 18/838,354

(22) PCT Filed: Feb. 22, 2023
(86) PCT No.: PCT/JP2023/006595

§ 371 Date: Aug. 14, 2024

(30) Foreign Application Priority Data
Mar. 3, 2022 (JP) 2022-032934

(51) Int. Cl. G16H 40/20

(52) U.S. Cl. CPC G16H 40/20

(57) ABSTRACT

Technology related to nursing care support is improved. A care management support method to be executed by an information processing apparatus includes acquiring a schedule of care for a person requiring nursing care for whom a predetermined level of nursing care is set, acquiring performance information on care for the person requiring nursing care, and presenting a suggestion for the person requiring nursing care based on information on the predetermined level of nursing care, the schedule, and the performance information on care.

### TECHNICAL FIELD

The present disclosure relates to a care management support method, an information processing apparatus, a system, and a program.

### BACKGROUND

Technology related to nursing care support has been developed. For example, Patent Literature (PTL) 1 discloses a visit planning device that minimizes the impact of changes to the schedule of planned nursing care visits to a subject, even when such changes are necessary, and enables more efficient visits to be carried out.

### CITATION LIST

### Patent Literature

PTL 1: JP 2014-149574 A

### SUMMARY

### Technical Problem

Although the technology in PTL 1 is related to improving efficiency by changing the schedule of nursing care visits, no particular consideration is made regarding improvement of the overall care management cycle, including the nursing care schedule. In other words, technology related to nursing care support has room for improvement.

In light of these circumstances, it is an aim of the present disclosure to improve technology related to nursing care support.

### Solution to Problem

A care management support method according to an embodiment of the present disclosure is a care management support method to be executed by an information processing apparatus, the care management support method including:
acquiring a schedule of care for a person requiring nursing care for whom a predetermined level of nursing care is set;
acquiring performance information on care for the person requiring nursing care; and
presenting a suggestion for the person requiring nursing care based on information on the predetermined level of nursing care, the schedule, and the performance information on care.

An information processing apparatus according to an embodiment of the present disclosure is an information processing apparatus including a controller, wherein
the controller is configured to
   acquire a schedule of care for a person requiring nursing care for whom a predetermined level of nursing care is set;
   acquire performance information on care for the person requiring nursing care; and
   present a suggestion for the person requiring nursing care based on information on the predetermined level of nursing care, the schedule, and the performance information on care.

A program according to an embodiment of the present disclosure is configured to cause a computer to execute operations including:
acquiring a schedule of care for a person requiring nursing care for whom a predetermined level of nursing care is set;
acquiring performance information on care for the person requiring nursing care; and
presenting a suggestion for the person requiring nursing care based on information on the predetermined level of nursing care, the schedule, and the performance information on care.

### Advantageous Effect

According to an embodiment of the present disclosure, technology related to nursing care support can be improved.

### BRIEF DESCRIPTION OF THE DRAWINGS

In the accompanying drawings:

FIG. 1 is a block diagram illustrating a schematic configuration of a system according to an embodiment of the present disclosure;

FIG. 2 is a block diagram illustrating a schematic configuration of a terminal apparatus;

FIG. 3 is a block diagram illustrating a schematic configuration of an information processing apparatus;

FIG. 4 is a diagram illustrating an example of a nursing care database;

FIG. 5 is a flowchart illustrating operations of the information processing apparatus according to an embodiment of the present disclosure;

FIG. 6 is a flowchart illustrating an example of a process to determine suggestion content;

FIG. 7 is a flowchart illustrating an example of a process to determine suggestion content for predetermined care;

FIG. 8 is a diagram illustrating an example of a user interface outputted by the terminal apparatus;

FIG. 9 is a diagram illustrating another example of a user interface outputted by the terminal apparatus;

FIG. 10 is a diagram illustrating an example of a user interface outputted by the terminal apparatus;

FIG. 11 is a diagram illustrating an example of a user interface outputted by the terminal apparatus;

FIG. 12 is a diagram illustrating an example of a user interface outputted by the terminal apparatus;

FIG. 13 is a diagram illustrating an example of a user interface outputted by the terminal apparatus; and

FIG. 14 is a diagram illustrating an example of an information collection sheet.

### DETAILED DESCRIPTION

A care management support system 1 according to an embodiment of the present disclosure is described below with reference to the drawings.

Identical or equivalent portions in the drawings are labeled with the same reference signs. In the explanation of the embodiments, a description of identical or equivalent portions is omitted or simplified as appropriate.

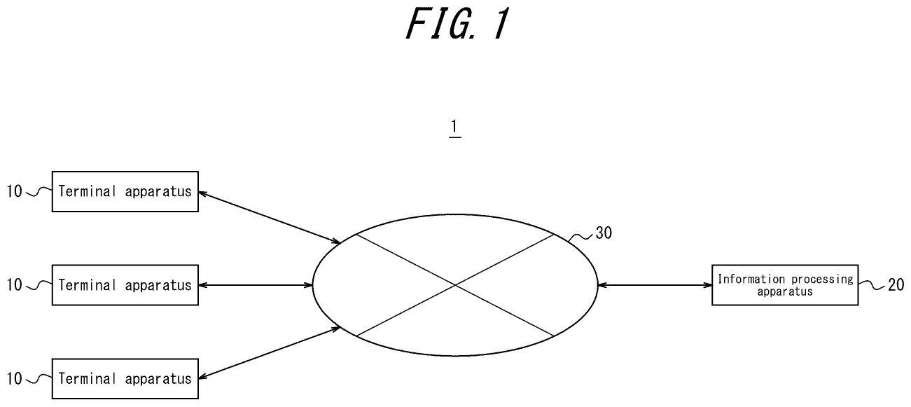

An outline and configuration of the care management support system 1 according to the present embodiment is described with reference to FIG. 1.

The care management support system 1 according to the present embodiment includes a plurality of terminal apparatuses 10 and an information processing apparatus 20. The plurality of terminal apparatuses 10 and the information processing apparatus 20 are communicably connected to a network 30, such as a mobile communication network, the Internet, and the like.

The plurality of terminal apparatuses 10 is any apparatus used by each user (such as a certified care worker, caregiver, care manager, or nurse). For example, a general-purpose electronic device, such as a smartphone or tablet terminal, or a dedicated electronic device can be adopted as the terminal apparatus 10. Although FIG. 1 illustrates an example of the care management support system 1 including three terminal apparatuses 10, this example is not limiting. The care management support system 1 may include fewer than three terminal apparatuses 10 or may include four or more terminal apparatuses 10.

The information processing apparatus 20 is, for example, a server apparatus installed in a data center or the like. For example, the information processing apparatus 20 is a server belonging to a cloud computing system or other computing system. The information processing apparatus 20 can communicate with the terminal apparatus 10 via the network 30. Although FIG. 1 illustrates an example of the care management support system 1 including one information processing apparatus 20, this example is not limiting. The care management support system 1 may include two or more information processing apparatuses 20.

First, an overview of the present embodiment is described, with details to be described later. The information processing apparatus 20 acquires information on a level of nursing care for a person requiring nursing care and a schedule of nursing care (hereinafter also referred to as “care”) for the person requiring nursing care. The information processing apparatus 20 also acquires performance information on care from a nursing care database. In the present embodiment, a person requiring nursing care is a person who needs nursing care or support in daily life.

Furthermore, the information processing apparatus 20 presents suggestions for the person requiring nursing care based on the information on the level of nursing care, the schedule, and the performance information on care. The suggestions include suggestions for improvement and warnings in care management related to the person requiring nursing care. For example, the suggestions include a proposal to change the classification of the level of nursing care set for the person requiring nursing care (hereinafter referred to as “consideration of classification change”). The information processing apparatus 20 presents the suggestions to the administrator or other users.

According to the present embodiment, the information processing apparatus 20 thus presents suggestions for the person requiring nursing care based on the information on the level of nursing care, the schedule, and the performance information on care. In other words, according to the present embodiment, technology related to nursing care support is improved in that suggestions for improvement pertaining to the care management cycle are determined by synthesizing information on the level of nursing care, the schedule, and performance information.

Next, each of the components of the care management support system 1 will be described in detail.

### (Configuration of Terminal Apparatus)

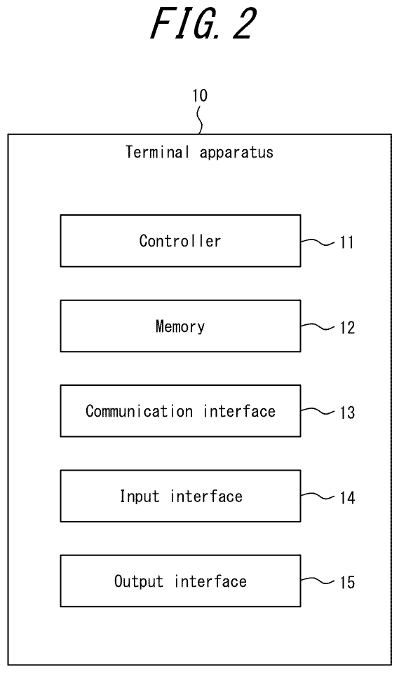

As illustrated in FIG. 2, the terminal apparatus 10 includes a controller 11, a memory 12, a communication interface 13, an input interface 14, and an output interface 15.

The controller 11 includes at least one processor, at least one dedicated circuit, or a combination thereof. The processor may, for example, be a general-purpose processor, such as a central processing unit (CPU) or graphics processing unit (GPU), or a dedicated processor specialized for particular processing. The dedicated circuit may, for example, be a field-programmable gate array (FPGA) or an application specific integrated circuit (ASIC). The controller 11 executes processing related to operations of the terminal apparatus 10 while controlling each component of the terminal apparatus 10.

The memory 12 includes at least one semiconductor memory, at least one magnetic memory, at least one optical memory, or a combination of at least two of these. The semiconductor memory is, for example, random access memory (RAM) or read only memory (ROM). The RAM is, for example, static random access memory (SRAM) or dynamic random access memory (DRAM). The ROM is, for example, electrically erasable programmable read only memory (EEPROM). The memory 12 functions as, for example, a main memory, an auxiliary memory, or a cache memory. The memory 12 stores data to be used for operation of the terminal apparatus 10 and data resulting from operation of the terminal apparatus 10.

The communication interface 13 includes at least one interface for communication with an external destination. The interface for communication may be an interface for wired communication or wireless communication. In the case of wired communication, the interface for communication may be a local area network (LAN) interface or a universal serial bus (USB), for example. In the case of wireless communication, the interface for communication may be an interface conforming to a mobile communication standard, such as Long Term Evolution (LTE), 4th Generation (4G), or 5th Generation (5G), or an interface conforming to short-range wireless communication such as Bluetooth® (Bluetooth is a registered trademark in Japan, other countries, or both). The communication interface 13 receives data for use in operation of the terminal apparatus 10 and transmits data resulting from operation of the terminal apparatus 10.

The input interface 14 includes at least one interface for input. The interface for input is, for example, a physical key, a capacitive key, a pointing device, or a touchscreen integrally provided with a display. The interface for input may, for example, be a microphone that receives audio input, a camera that accepts gesture input, or the like. The input interface 14 receives an operation for inputting data used in operation of the terminal apparatus 10. Instead of being provided in the terminal apparatus 10, the input interface 14 may be connected to the terminal apparatus 10 as an external input device. Any appropriate connection method can be used, such as USB, High-Definition Multimedia Interface (HDMI®) (HDMI is a registered trademark in Japan, other countries, or both), or Bluetooth®.

The output interface 15 includes at least one interface for output. The interface for output is, for example, a display that outputs information as images, a speaker that outputs information as audio, or the like. The display may, for example, be a liquid crystal display (LCD) or an organic electro luminescence (EL) display. The output interface 15 displays and outputs data resulting from operation of the terminal apparatus 10. Instead of being provided in the terminal apparatus 10, the output interface 15 may be connected to the terminal apparatus 10 as an external output device. Any appropriate connection method can be used, such as USB, HDMI®, or Bluetooth®.

The functions of the terminal apparatus 10 are implemented by a processor corresponding to the terminal apparatus 10 executing a program according to the present embodiment. In other words, the functions of the terminal apparatus 10 are implemented by software. The program causes a computer to function as the terminal apparatus 10 by causing the computer to execute the operations of the terminal apparatus 10. In other words, the computer functions as the terminal apparatus 10 by executing the operations of the terminal apparatus 10 according to the program.

The program according to the present embodiment can be recorded on a computer readable recording medium. Computer readable recording media include non-transitory computer readable recording media, examples of which are a magnetic recording apparatus, an optical disc, a magneto-optical recording medium, and a semiconductor memory. The program is, for example, distributed by the sale, transfer, or lending of a portable recording medium such as a digital versatile disk (DVD) or a compact disk read only memory (CD-ROM) on which the program is recorded. The program may also be distributed by storing the program in the storage of an external server and transmitting the program from the external server to another computer. The program may also be provided as a program product.

A portion or all of the functions of the terminal apparatus 10 may be implemented by a dedicated circuit corresponding to the controller 11. In other words, a portion or all of the functions of the terminal apparatus 10 may be implemented by hardware.

### (Configuration of Information Processing Apparatus)

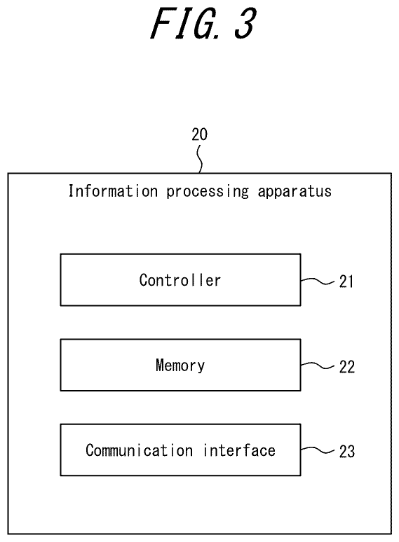

As illustrated in FIG. 3, the information processing apparatus 20 includes a controller 21, a memory 22, and a communication interface 23.

The controller 21 includes at least one processor, at least one dedicated circuit, or a combination thereof. The processor may be a general-purpose processor, such as a CPU or GPU, or a dedicated processor specialized for particular processing. The dedicated circuit is, for example, an FPGA or an ASIC. The controller 21 executes processing related to operation of the information processing apparatus 20 while controlling each component of the information processing apparatus 20.

The memory 22 includes at least one semiconductor memory, at least one magnetic memory, at least one optical memory, or a combination of at least two of these. The semiconductor memory is, for example, RAM or ROM. The RAM is, for example, SRAM or DRAM. The ROM is, for example, EEPROM. The memory 22 functions as, for example, a main memory, an auxiliary memory, or a cache memory. The memory 22 stores data to be used for operation of the information processing apparatus 20 and data resulting from operation of the information processing apparatus 20.

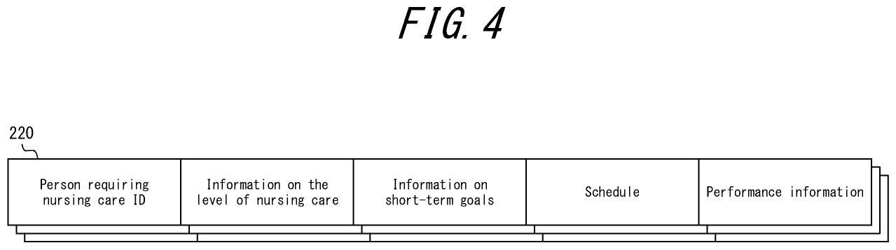

In the present embodiment, the memory 22 stores a nursing care database 220. FIG. 4 illustrates an example of the nursing care database 220. For example, the nursing care database 220 includes a person requiring nursing care ID, information on the level of nursing care, information on short-term goals, a schedule, and performance information.

The person requiring nursing care ID is information that uniquely identifies the person requiring nursing care. For example, the person requiring nursing care ID may be a number, a symbol, or a combination thereof, assigned to a resident of a nursing home, elderly care facility, or other residential facility. An example in which the person requiring nursing care is a resident of a residential facility is described in the present embodiment, but the person requiring nursing care is not limited to this case. For example, the person requiring nursing care may be a person who receives visiting nursing care.

The information on the level of nursing care is information determined by the level of nursing care. The level of nursing care is an indicator of the degree of nursing care or support needed in daily life. In the present embodiment, the level of nursing care can be evaluated at seven levels: nursing care level 1 to 5, and support level 1 to 2. The level of nursing care for each person requiring nursing care is a predetermined level of nursing care that is set based on an assessment of the person requiring nursing care. The information on the level of nursing care is information determined based on the predetermined level of nursing care set in this way. For example, the information on the level of nursing care may include a first reference time. The first reference time is a reference assistance time corresponding to a level of nursing care that is one level higher than the predetermined level of nursing care. The information on the level of nursing care may, for example, include a second reference time. The second reference time is a reference assistance time corresponding to the predetermined level of nursing care. The information on the level of nursing care may, for example, include a third reference time. The third reference time is a reference assistance time for each type of prescribed care (hereinafter also referred to as predetermined care) in a predetermined level of nursing care. The predetermined care includes care pertaining to toilet use, mobility, drinking, taking medication, grooming and cleanliness, housework, and bathing. In the present embodiment, the information on the level of nursing care is described as including the first reference time, the second reference time, and the third reference time.

The information on the short-term goals includes information on the content and the end date of the short-term goals. The short-term goals are specific goals set for each person requiring nursing care based on a care plan. The end date of a short-term goal is the date by which the short-term goal is to be achieved.

The schedule is information about the schedule of care set for each person requiring nursing care over a predetermined period of time. The predetermined period of time is, for example, two weeks. The schedule includes, for example, information on the type and content of care scheduled to be provided over the next two weeks, the number of cases of care, and the date and time when the care is scheduled to be provided. The schedule is determined based on, for example, a reference number of times care is provided according to the level of nursing care of the person requiring nursing care. The schedule is adjusted as necessary based on the past performance of care for the person requiring nursing care and requests from the person requiring nursing care or the like.

The performance information is any information on the performance of care provided to each person requiring nursing care during a predetermined period of time. The predetermined period of time is, for example, two weeks. The performance information includes not only the performance of care that was on the schedule in advance and was performed, but also the performance of care that was not on the schedule in advance (hereinafter also referred to as “unscheduled care”). In other words, unscheduled care is care that was not originally on the schedule. For example, unscheduled care includes toilet use care provided on an emergency basis.

At least a portion of the data in the nursing care database 220 is created or edited based on user input to the plurality of terminal apparatuses 10 or the information processing apparatus 20. At least some data in the nursing care database 220 may be automatically created or edited by a measurement apparatus such as a wearable device held by the person requiring nursing care.

The communication interface 23 includes at least one interface for communication with an external destination. The interface for communication may be an interface for wired communication or wireless communication. In the case of wired communication, the interface for communication may be a LAN interface or a USB, for example. In the case of wireless communication, the interface for communication may be an interface conforming to a mobile communication standard, such as LTE, 4G, or 5G, or an interface conforming to short-range wireless communication such as Bluetooth®. The communication interface 23 receives data for use in operation of the information processing apparatus 20 and transmits data resulting from operation of the information processing apparatus 20.

The functions of the information processing apparatus 20 are implemented by a processor corresponding to the controller 21 executing a program according to the present embodiment. In other words, the functions of the information processing apparatus 20 are implemented by software. The program causes a computer to function as the information processing apparatus 20 by causing the computer to execute the operations of the information processing apparatus 20. In other words, the computer functions as the information processing apparatus 20 by executing the operations of the information processing apparatus 20 in accordance with the program.

The computer in the present embodiment temporarily stores, in the main memory, the program recorded on a portable recording medium or transferred from a server, for example. The computer uses a processor to read the program stored in the main memory and executes processing with the processor in accordance with the read program. The computer may read the program directly from the portable recording medium and execute processing in accordance with the program. Each time the program is received from an external server, the computer may sequentially execute processing in accordance with the received program. Processing may be executed by an application service provider (ASP) type of service that implements functions only via execution instructions and result acquisition, without transmission of the program from an external server to the computer. Examples of the program include an equivalent to the program represented as information provided for processing by an electronic computer. For example, data that is not a direct command for a computer but that has the property of specifying processing by the computer corresponds to the “equivalent to the program”.

A portion or all of the functions of the information processing apparatus 20 may be implemented by a dedicated circuit corresponding to the controller 21. In other words, a portion or all of the functions of the information processing apparatus 20 may be implemented by hardware.

### (Operations of Information Processing Apparatus)

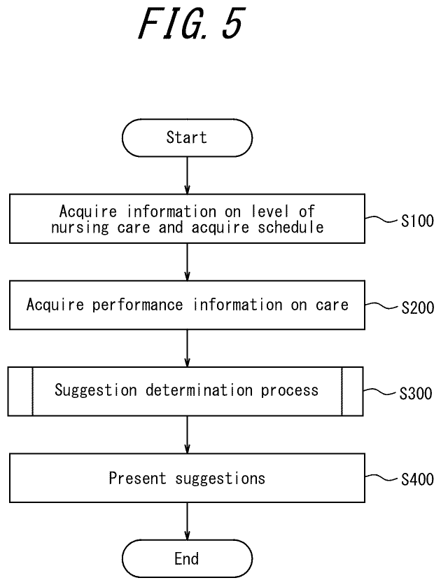

With reference to FIG. 5, operations of the information processing apparatus 20 according to the present embodiment are now described. FIG. 5 is a flowchart illustrating an example of a method executed by the information processing apparatus 20 according to the present embodiment.

Step S100: the controller 21 of the information processing apparatus 20 acquires information on the level of nursing care for each person requiring nursing care and the schedule of care for each person requiring nursing care from the nursing care database 220.

Step S200: the controller 21 acquires performance information on care for each person requiring nursing care from the nursing care database 220.

Step S300: the controller 21 determines suggestions to present to the user based on the information on the level of nursing care, the schedule, and the performance information on care for the person requiring nursing care.

Step S400: the controller 21 presents the content of the suggestions determined in step S300. In other words, the controller 21 presents suggestions for the person requiring nursing care based on the information on the level of nursing care, the schedule, and the performance information on nursing care for the person requiring nursing care.

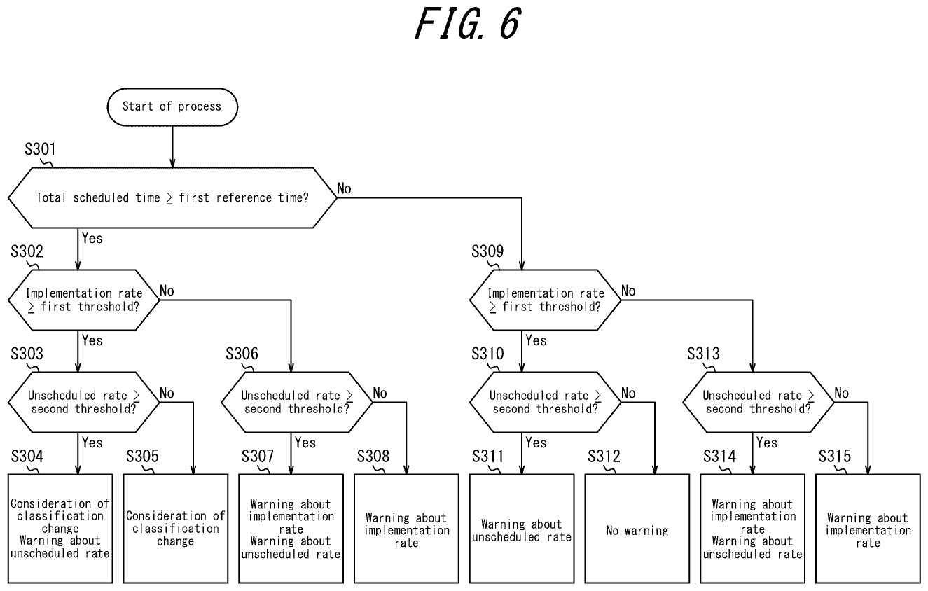

The determination of the content of suggestions pertaining to a person requiring nursing care can be made by various methods. FIG. 6 is a flowchart illustrating an example of a process to determine suggestion content.

Step S301: the controller 21 of the information processing apparatus 20 determines whether the total time of the schedule of nursing care is equal to or greater than the first reference time. In a case in which the total time of the schedule of nursing care is equal to or greater than the first reference time, the process proceeds to step S302. Conversely, in a case in which the total time of the schedule of nursing care is less than the first reference time, the process proceeds to step S309.

Step S302: the controller 21 determines whether an implementation rate is equal to or greater than a first threshold. The implementation rate is an index determined by the ratio of the total number of cases of care based on the performance information to the number of cases in the schedule. In other words, the implementation rate indicates the percentage of scheduled care that was actually implemented. For example, the implementation rate is determined by Equation (1) below.

(implementation rate)=(total number of cases of care based on performance information)/(total number of cases of care based on schedule) (1)

The method of determining the implementation rate is not limited to this example. For example, in Equation (1), the implementation rate is determined by the ratio of the number of cases, but the implementation rate may also be determined by the ratio of the total time of care based on implementation information to the total time of care based on the schedule.

In a case in which the implementation rate is equal to or greater than the first threshold, the process proceeds to step S303. Conversely, in a case in which the implementation rate is less than the first threshold, the process proceeds to step S306.

Step S303: the controller 21 determines whether an unscheduled rate is equal to or greater than a second threshold. The unscheduled rate is an index determined by the ratio of the total number of unscheduled cases of care among the total number of cases of care based on the performance information to the number of cases in the schedule. In other words, the unscheduled rate is an index that indicates how much unexpected or urgent care was performed relative to the scheduled cases of care. For example, the unscheduled rate is determined by Equation (2) below.

(unscheduled rate)=(total number of unscheduled cases of care based on performance information)/(total number of cases of care based on schedule) (2)

The method of determining the unscheduled rate is not limited to this example. For example, in Equation (2), the unscheduled rate is determined by the ratio of the number of cases, but the unscheduled rate may also be determined by the ratio of the total time of care based on implementation information to the total time of care based on the schedule.

In a case in which the unscheduled rate is equal to or greater than the second threshold, the process proceeds to step S304. Conversely, in a case in which the unscheduled rate is less than the second threshold, the process proceeds to step S305.

Step S304: the controller 21 determines, as suggestions, a consideration of classification change and a warning that the unscheduled rate is equal to or greater than the reference.

Step S305: in a case in which the unscheduled rate is less than the second threshold in step S303, the controller 21 determines, as a suggestion, a consideration of classification change.

Step S306: in a case in which the implementation rate is less than the first threshold in step S302, the controller 21 determines whether the unscheduled rate is equal to or greater than the second threshold. In a case in which the unscheduled rate is equal to or greater than the second threshold, the process proceeds to step S307. Conversely, in a case in which the unscheduled rate is less than the second threshold, the process proceeds to step S308.

Step S307: the controller 21 determines, as suggestions, a warning that the implementation rate is less than the reference and a warning that the unscheduled rate is equal to or greater than the reference.

Step S308: in a case in which the unscheduled rate is less than the second threshold in step S306, the controller 21 determines, as a suggestion, a warning that the implementation rate is less than the reference.

Step S309: in a case in which the total time of scheduled care in step S301 is less than the first reference time, the controller 21 determines whether the implementation rate of care is equal to or greater than the first threshold. In a case in which the implementation rate is equal to or greater than the first threshold, the process proceeds to step S310. Conversely, in a case in which the implementation rate is less than the first threshold, the process proceeds to step S313.

Step S310: the controller 21 determines whether the unscheduled rate is equal to or greater than the second threshold. In a case in which the unscheduled rate is equal to or greater than the second threshold, the process proceeds to step S311. Conversely, in a case in which the unscheduled rate is less than the second threshold, the process proceeds to step S312.

Step S311: the controller 21 determines, as a suggestion, a warning that the unscheduled rate is equal to or greater than the reference.

Step S312: in a case in which the unscheduled rate is less than the second threshold in step S310, the controller 21 determines Null as a suggestion. In other words, in this case, since the first reference time, the implementation rate, and the unscheduled rate all satisfy the reference, no warning or other special action is performed.

Step S313: in a case in which the implementation rate is less than the first threshold in step S309, the controller 21 determines whether the unscheduled rate is equal to or greater than the second threshold. In a case in which the unscheduled rate is equal to or greater than the second threshold, the process proceeds to step S314. Conversely, in a case in which the unscheduled rate is less than the second threshold, the process proceeds to step S315.

Step S314: the controller 21 determines, as suggestions, a warning that the implementation rate is less than the reference and a warning that the unscheduled rate is equal to or greater than the reference.

Step S315: in a case in which the unscheduled rate is less than the second threshold in step S313, the controller 21 determines, as a suggestion, a warning that the implementation rate is less than the reference.

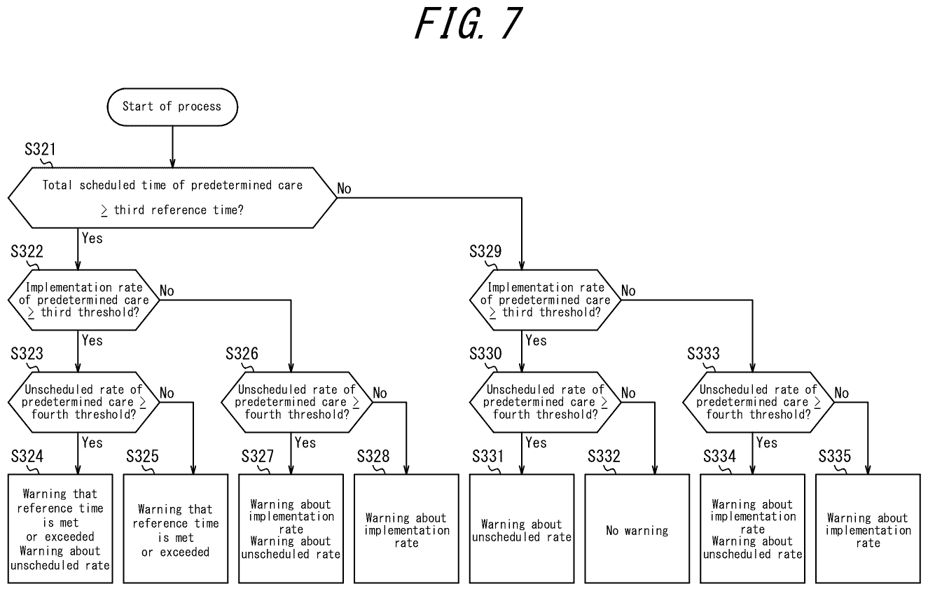

In the process of determining the content of suggestions in FIG. 6, the controller 21 of the information processing apparatus 20 may determine the content of suggestions pertaining to each instance of predetermined care. FIG. 7 is a flowchart illustrating an example of a process to determine suggestion content pertaining to each instance of predetermined care.

Step S321: the controller 21 of the information processing apparatus 20 determines whether the total scheduled time of predetermined care is equal to or greater than a third reference time. In a case in which the total scheduled time of predetermined care is equal to or greater than the third reference time, the process proceeds to step S322. Conversely, in a case in which the total scheduled time of predetermined care is less than the third reference time, the process proceeds to step S329.

Step S322: the controller 21 determines whether an implementation rate of predetermined care is equal to or greater than a third threshold. The third threshold may be the same as or different from the first threshold. The implementation rate of predetermined care is an index determined by the ratio of the total number of cases of predetermined care based on the performance information to the number of cases in the schedule for predetermined care. In other words, the implementation rate of predetermined care indicates the percentage of scheduled predetermined care that was actually implemented. For example, the implementation rate of predetermined care is determined by Equation (3) below.

(implementation rate of predetermined care)=(total number of cases of predetermined care based on performance information)/(total number of cases of predetermined care based on schedule) (3)

The method of determining the implementation rate of predetermined care is not limited to this example. For example, in Equation (3), the implementation rate of predetermined care is determined by the ratio of the number of cases, but the implementation rate of predetermined care may also be determined by the ratio of the total time of predetermined care based on implementation information to the total time of predetermined care based on the schedule.

In a case in which the implementation rate of predetermined care is equal to or greater than the third threshold, the process proceeds to step S323. Conversely, in a case in which the implementation rate of predetermined care is less than the third threshold, the process proceeds to step S326.

Step S323: the controller 21 determines whether an unscheduled rate of predetermined care is equal to or greater than a fourth threshold. The fourth threshold may be the same as or different from the second threshold. The unscheduled rate of predetermined care is an index determined by the ratio of the total number of cases of predetermined care that were unscheduled among the total number of cases of predetermined care based on the performance information to the number of cases in the schedule. In other words, the unscheduled rate is an index that indicates how much unexpected or urgent predetermined care was performed relative to the scheduled cases of predetermined care. For example, the unscheduled rate of predetermined care is determined by Equation (4) below.

(unscheduled rate of predetermined care)=(total number of unscheduled cases of predetermined care based on performance information)/(total number of cases of predetermined care based on schedule) (4)

The method of determining the unscheduled rate of predetermined care is not limited to this example. For example, in Equation (4), the unscheduled rate is determined by the ratio of the number of cases, but the unscheduled rate of predetermined care may also be determined by the ratio of the total time of unscheduled predetermined care based on implementation information to the total time of predetermined care based on the schedule.

In a case in which the unscheduled rate of predetermined care is equal to or greater than the fourth threshold, the process proceeds to step S324. Conversely, in a case in which the unscheduled rate of predetermined care is less than the fourth threshold, the process proceeds to step S325.

Step S324: the controller 21 determines, as a suggestion, a warning that the predetermined care is equal to or greater than the reference time and that the unscheduled rate is equal to or greater than the reference.

Step S325: in a case in which the unscheduled rate is less than the fourth threshold in step S323, the controller 21 determines, as a suggestion, a warning that the predetermined care is equal to or greater than the reference time.

Step S326: in a case in which the implementation rate of predetermined care is less than the third threshold in step S322, the controller 21 determines whether the unscheduled rate of predetermined care is equal to or greater than the fourth threshold. In a case in which the unscheduled rate of predetermined care is equal to or greater than the fourth threshold, the process proceeds to step S327. Conversely, in a case in which the unscheduled rate of predetermined care is less than the fourth threshold, the process proceeds to step S328.

Step S327: the controller 21 determines, as suggestions, a warning that the implementation rate of predetermined care is less than the reference and a warning that the unscheduled rate of predetermined care is equal to or greater than the reference.

Step S328: in a case in which the unscheduled rate is less than the fourth threshold in step S326, the controller 21 determines, as a suggestion, a warning that the implementation rate of predetermined care is less than the reference.

Step S329: in a case in which the total time of scheduled predetermined care in step S321 is less than the third reference time, the controller 21 determines whether the implementation rate of predetermined care is equal to or greater than the third threshold. In a case in which the implementation rate of predetermined care is equal to or greater than the third threshold, the process proceeds to step S330. Conversely, in a case in which the implementation rate of predetermined care is less than the third threshold, the process proceeds to step S333.

Step S330: the controller 21 determines whether an unscheduled rate of predetermined care is equal to or greater than a fourth threshold. In a case in which the unscheduled rate of predetermined care is equal to or greater than the fourth threshold, the process proceeds to step S331. Conversely, in a case in which the unscheduled rate of predetermined care is less than the fourth threshold, the process proceeds to step S332.

Step S331: the controller 21 determines, as a suggestion, a warning that the unscheduled rate of predetermined care is equal to or greater than the reference.

Step S332: in a case in which the unscheduled rate is less than the fourth threshold in step S330, the controller 21 determines Null as a suggestion. In other words, in this case, since the third reference time, the implementation rate of predetermined care, and the unscheduled rate of predetermined care all satisfy the reference, no warning or other special action is performed.

Step S333: in a case in which the implementation rate of predetermined care is less than the third threshold in step S329, the controller 21 determines whether the unscheduled rate of predetermined care is equal to or greater than the fourth threshold. In a case in which the unscheduled rate of predetermined care is equal to or greater than the fourth threshold, the process proceeds to step S334. Conversely, in a case in which the unscheduled rate of predetermined care is less than the fourth threshold, the process proceeds to step S335.

Step S334: the controller 21 determines, as suggestions, a warning that the implementation rate of predetermined care is less than the reference and a warning that the unscheduled rate of predetermined care is equal to or greater than the reference.

Step S335: in a case in which the unscheduled rate is less than the fourth threshold in step S333, the controller 21 determines, as a suggestion, a warning that the implementation rate of predetermined care is less than the reference.

Here, any method can be employed for presenting suggestions in step S400. In the present embodiment, an example of visually presenting suggestions via a user interface that includes the suggestions will be described. Such a user interface may be provided by the terminal apparatus 10 accessing the information processing apparatus 20 or a web server via browser software. Alternatively, an application according to the care management support system 1 may be installed in the terminal apparatus 10, and the user interface may be provided by the terminal apparatus 10 communicating as appropriate with the information processing apparatus 20 or an application server via the application. In this case, an authentication process or the like pertaining to the user may be performed by the terminal apparatus 10 and the information processing apparatus 20, or the terminal apparatus 10 and the web server, as appropriate.

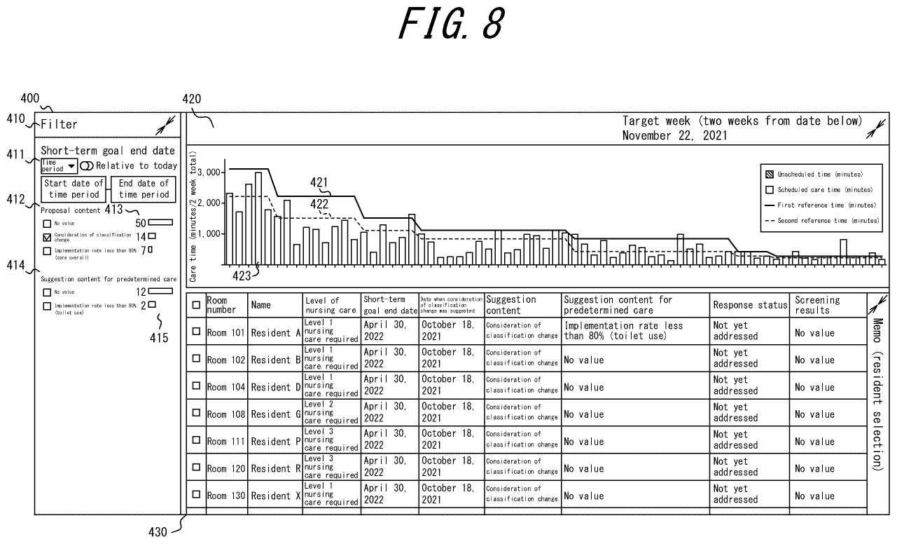

FIG. 8 illustrates an example of a user interface displayed by the output interface 15 of the terminal apparatus 10. FIG. 8 illustrates a user interface 400 provided to a user of a certain residential facility as an example. As illustrated in FIG. 8, the user interface 400 includes an object 410, a graph 420, and a table 430. The object 410 is an object that provides a filter function based on user input. The graph 420 illustrates information on a plurality of persons requiring nursing care. The table 430 illustrates various information including suggestions for each person requiring nursing care.

The object 410 includes objects 411 to 415. The object 411 provides a filtering function based on time period. The object 411 illustrated in FIG. 8 as an example includes a selection field, a toggle switch, and an input field. By manipulating the object 411, the user can specify, in days, weeks, months, or the like, the time period for statistical information displayed in the graph 420. The user can also specify the start and end of the time period by, for example, inputting the start and end dates of the time period in the input fields of the object 411. In FIG. 8, the object 411 has not yet received input, and no time period has been specified. In a case in which no time period is specified by the object 411, information on a default time period is displayed. The default time period is, for example, the time period spanning the previous two weeks. The graph 420 in FIG. 8 illustrates information for the time period from Nov. 22, 2021 until two weeks later.

The object 412 provides a filtering function based on suggestion content. The object 412 illustrated in FIG. 8 as an example includes a check box for each type of suggestion and an object 413 with a bar graph for each type of suggestion. By manipulating the object 412, the user can filter the information, displayed in the table 430, on persons requiring nursing care so as to display only information associated with a specific type of suggestion. Here, in FIG. 8, “Consideration of classification change” is selected. In this case, the information displayed in the table 430 is limited to only information that includes “consideration of classification change” in the suggestion content. The object 413 indicates the number of cases for each type of suggestion. By confirming the object 413, the user can easily compare the number of cases of each type of suggestion.

The object 414 provides a filtering function to filter by suggestion content pertaining to predetermined care. The object 414 illustrated in FIG. 8 as an example includes a check box for each type of suggestion and an object 415 with a bar graph for each type of suggestion. By manipulating the object 414, the user can filter the information, displayed in the table 430, on persons requiring nursing care so as to display only information associated with a specific type of suggestion pertaining to predetermined care. Here, in FIG. 8, none of the check boxes is selected. In this case, the information displayed in table 430 is not filtered based on the suggestion content pertaining to the predetermined care, but rather all cases are displayed. The object 415 indicates the number of cases for each type of suggestion pertaining to predetermined care. By confirming the object 415, the user can easily compare the number of cases of each type of suggestion pertaining to predetermined care.

The graph 420 includes a graph 421 pertaining to the first reference time, a graph 422 pertaining to the second reference time, and a bar graph 423 pertaining to the scheduled care time, for each person requiring nursing care. The horizontal axis of the graph 420 indicates each person requiring nursing care, and the vertical axis indicates the time pertaining to care. The graph 420 enables the user easily to compare the scheduled care time for each person requiring nursing care with the first reference time and the second reference time. In FIG. 8, an example is illustrated in which the name of each person requiring nursing care is not displayed in the graph 420, but this configuration is not limiting. The name of each person requiring nursing care may be displayed on the horizontal axis of the graph 420.

The table 430 includes the room number, name, level of nursing care, short-term goal end date, date when consideration of classification change was suggested, suggestion content, and suggestion content for predetermined care. The room number, name, level of nursing care, and short-term goal end date are generated based on the nursing care database 220. The date when consideration of classification change was suggested, the suggestion content, and the suggestion content for predetermined care are, for example, generated by the processes according to the flowcharts in FIGS. 5 to 7. Specifically, the date when consideration of classification change was suggested is determined based on the date and time when consideration of classification change was determined as a suggestion in step S300 of FIG. 5. The suggestion content and the suggestion content for predetermined care are determined based on the suggestion determination processes illustrated in FIGS. 6 and 7, respectively. In other words, the care management support method in the present embodiment can display a list of suggestions corresponding to each of a plurality of persons requiring nursing care. In this way, measures for a plurality of persons requiring nursing care can be grasped in a timely manner.

As illustrated in FIG. 8, the table 430 may include response status and screening results. These pieces of information are associated with the suggestions and suggestions for predetermined care. In a case in which the table 430 includes these pieces of information, the corresponding information is stored in the nursing care database 220 for each person requiring nursing care.

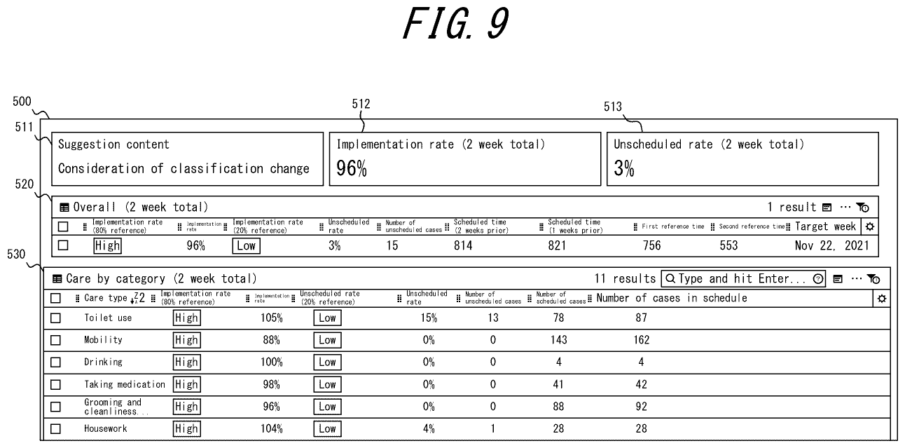

FIG. 9 illustrates another example of a user interface displayed by the output interface 15 of the terminal apparatus 10. FIG. 9 is a user interface 500 pertaining to information on individual persons requiring nursing care. This user interface 500 may, for example, be provided in a case in which a specific person in need of nursing care displayed by the table 430 in FIG. 8 is selected by clicking, tapping, or the like.

The user interface 500 includes objects 511 to 513, a table 520, and a table 530. The object 511 displays suggestions pertaining to persons requiring nursing care and suggestions for predetermined care. The object 512 displays the implementation rate of care for the person requiring nursing care. The object 513 displays the unscheduled rate of care for the person requiring nursing care.

The table 520 illustrates detailed information on the overall schedule and performance of care for persons requiring nursing care. The table 520, illustrated in FIG. 9, includes information on the degree of the implementation rate, the value of the implementation rate, the degree of the unscheduled rate, the value of the unscheduled rate, the number of unscheduled cases, the scheduled time (2 weeks prior), the scheduled time (1 week prior), the first reference time, the second reference time, and the target week. The degree of the implementation rate is either “high” or “low”. The degree of the implementation rate is displayed as “high” in a case in which the implementation rate is equal to or greater than the first threshold and “low” in a case in which the implementation rate is less than the first threshold. The degree of the unscheduled rate is either “high” or “low”. The degree of the unscheduled rate is displayed as “high” in a case in which the unscheduled rate is equal to or greater than the second threshold and “low” in a case in which the unscheduled rate is less than the second threshold.

The table 530 illustrates detailed information on the schedule and performance of predetermined care for persons requiring nursing care. The table 530 illustrated in FIG. 9 includes information on the degree of the implementation rate, the implementation rate, the degree of the unscheduled rate, unscheduled rate, the number of unscheduled cases, and the number of cases in the schedule for each type of predetermined care. The degree of the implementation rate of predetermined care is either “high” or “low”. The degree of the implementation rate is displayed as “high” in a case in which the implementation rate is equal to or greater than the third threshold and “low” in a case in which the implementation rate is less than the third threshold. The degree of the unscheduled rate of predetermined care is either “high” or “low”. The degree of the unscheduled rate is displayed as “high” in a case in which the unscheduled rate is equal to or greater than the fourth threshold and “low” in a case in which the unscheduled rate is less than the fourth threshold.

The user interface 500 enables the user easily to grasp the suggestions for each person requiring nursing care, and to grasp in detail the schedule and performance information that are the basis for the suggestions.

As described above, according to the present embodiment, the information processing apparatus 20 presents suggestions for the person requiring nursing care based on the information on the level of nursing care, the schedule, and the performance information on care. In other words, according to the present embodiment, technology related to nursing care support is improved in that suggestions for improvement pertaining to the care management cycle are determined by synthesizing information on the level of nursing care, the schedule, and performance information.

According to the present embodiment, the information on the level of nursing care includes the first reference time, i.e., the reference assistance time corresponding to a level of nursing care higher than the predetermined level of nursing care set for the person requiring nursing care. Therefore, appropriate proposals can be presented when care is provided to a degree exceeding the level of nursing care set for the person requiring nursing care.

In the present embodiment, the performance information includes the implementation rate. In other words, according to the present embodiment, suggestions are presented based on whether care was actually provided to the person requiring nursing care, thus making it possible to present more realistic suggestions.

In the present embodiment, the performance information includes the unscheduled rate. In other words, according to the present embodiment, suggestions are presented based on the degree to which care for persons requiring nursing care is performed unexpectedly. Since unexpected care is highly likely to decrease productivity, proposals to prevent the decrease in productivity can be presented by presenting suggestions based on the unscheduled rate.

In the present embodiment, an example in which the suggestions are at least one of a change in classification of level of nursing care, a warning pertaining to the implementation rate, a warning pertaining to the unscheduled rate, and a warning pertaining to predetermined care has been illustrated, but this configuration is not limiting. The suggestions may, for example, include, a warning pertaining to a swing and miss rate. The swing and miss rate is the ratio of the number of cases of care that were scheduled and for which a user such as the care staff got prepared, but which were not actually performed because the care was no longer needed (hereinafter referred to as “swing and miss care”), to the number of cases of scheduled care. In the case of a warning pertaining to the swing and miss rate, the performance information includes the swing and miss rate. Since swing and miss care is highly likely to decrease productivity, proposals to prevent the decrease in productivity can be presented by presenting suggestions based on the swing and miss rate. The suggestions may, for example, include, a warning pertaining to a declination rate. The declination rate is the ratio of the number of cases of care that were scheduled and for which a user such as the care staff got prepared, but which were not actually performed because the care was declined, refused, or rejected by the person requiring nursing care or the like (herein after referred to as “declined care”), to the number of cases of scheduled care. In the case of a warning pertaining to the declination rate, the performance information includes the declination rate. Since declined care is highly likely to decrease productivity, proposals to prevent the decrease in productivity can be presented by presenting suggestions based on the declination rate. Additionally, in a case in which the implementation rate is lower than a predetermined value (for example, a predetermined value lower than the first threshold), there is a risk that data on the performed care has not been inputted, or that data has been inputted incorrectly. For example, in such a case, the care management support system 1 may present a warning about data entry error as a suggestion.

The controller 21 of the information processing apparatus 20 may present a status pertaining to an action corresponding to the suggestion. An action corresponding to a suggestion is a specific action to be taken based on the suggestion. For example, in a case in which the suggestion is a consideration of classification change, the actions corresponding to such a suggestion include “classification change determination”, “care recipient/family member confirmation”, and “application”. The classification change determination is an action to determine whether it is appropriate to consider changing the classification of the person requiring nursing care. This action is performed by the user, such as a care manager, based on a comprehensive determination including, for example, the various conditions of the person requiring nursing care. The “care recipient/family member confirmation” is an action to confirm, with the care recipient or concerned party, the change in classification of the level of nursing care of the person requiring nursing care. This action is performed by the user, such as a care manager, with respect to at least one of the person requiring nursing care and a family member. The “application” is the action of preparing documents, mailing them, and so forth to change the level of nursing care of a person requiring nursing care. This action is performed by a user such as a caregiver. For example, the process is executed in the order “classification change determination”, “care recipient/family member confirmation”, and “application”. In such a process, the execution status of each action is preferably easily grasped. To that end, the controller 21 may present the status pertaining to the action corresponding to the suggestion.

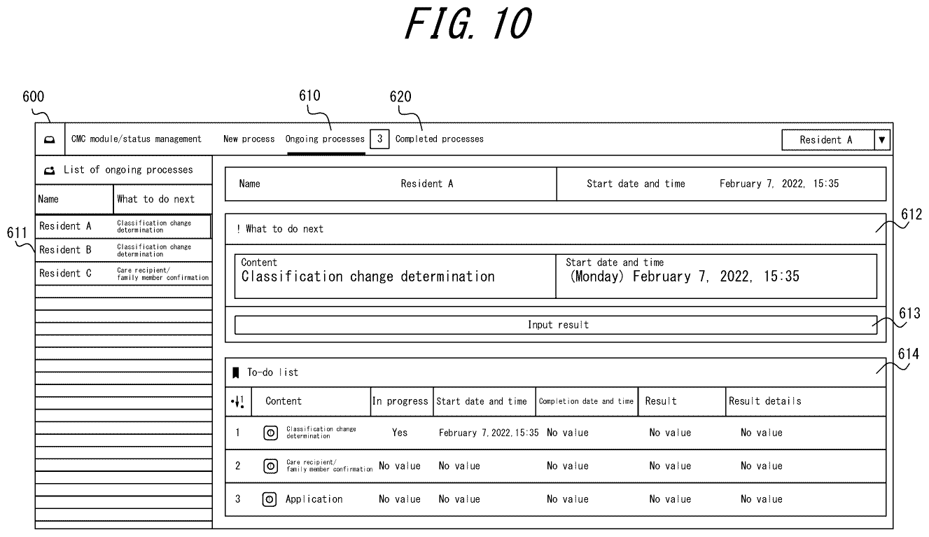

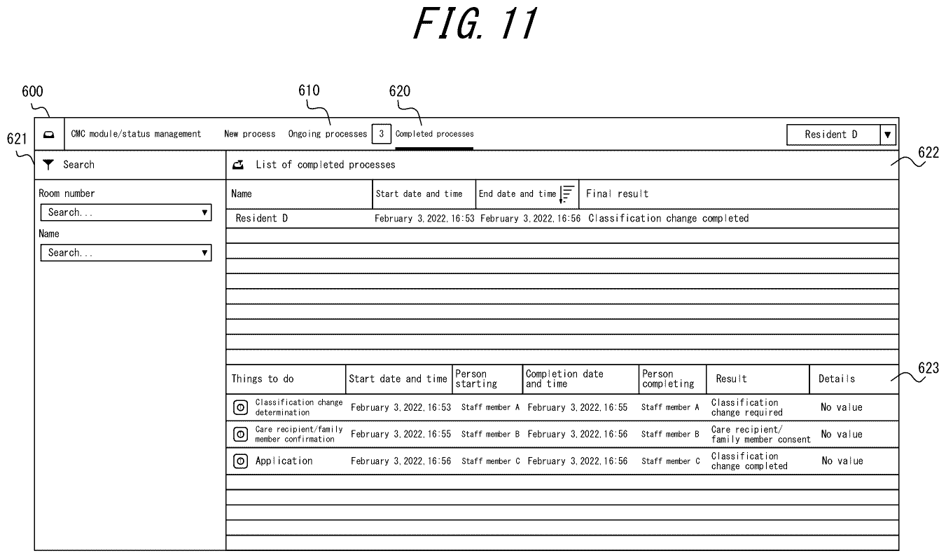

FIGS. 10 and 11 are diagrams illustrating an example of the output interface 15 of the terminal apparatus 10 displaying the status pertaining to the action corresponding to the suggestion presented by the controller 21. As illustrated in FIGS. 10 and 11, the status pertaining to the action corresponding to the suggestion is displayed by the user interface. The user interface 600 illustrated in FIG. 10 includes a tab 610 and a tab 620. The tab 610 and tab 620 are objects that switch the information displayed on the user interface 600. When the tab 610 is selected by clicking or the like, information pertaining to ongoing processes is displayed by the user interface 600. On the other hand, when the tab 620 is selected by clicking or the like, information pertaining to completed processes is displayed by the user interface 600. FIGS. 10 and 11 illustrate the user interface 600 in the case of the tab 610 being selected by clicking or the like, and the case of the tab 620 being selected by clicking or the like, respectively. The information pertaining to the ongoing processes includes the status of incomplete actions among the information on any person requiring nursing care selected by the user as a management target (hereinafter also referred to as a person requiring nursing care who is targeted for process management). In other words, the information pertaining to the ongoing processes includes the progress status of processes for the person requiring nursing care who is targeted for process management.

The user interface 600 of FIG. 10 includes a list of ongoing processes 611, selected person information 612, an input button 613, and a to-do list 614. The list of ongoing processes 611 is a tabular object in which the persons requiring nursing care for whom actions still need to be taken, among the persons requiring nursing care who are targeted for process management, are associated with the next action to be taken. In the list of ongoing processes 611 illustrated in FIG. 10, the persons requiring nursing care for whom actions still need to be taken, among the persons requiring nursing care who are targeted for process management, are residents A to C. The next actions to be taken for residents A to C are classification change determination, classification change determination, and care recipient/family member confirmation, respectively.

The selected person information 612 is information on the person requiring nursing care who is selected by clicking or the like from the list of ongoing processes 611. In FIG. 10, resident A is selected, and information on resident A is displayed. The selected person information 612 includes the next action to be taken and the start date on which the action was performed. The input button 613 is an object for the user to input the status pertaining to the action corresponding to the suggestion. The status pertaining to the action includes the start date and time of the action, the progress status, the completion date and time, and the result details. The result details include the determination result, the confirmation result, the acceptance result, and the reason for these results, for each action. In other words, the input button 613 collects various information pertaining to the status of each action.

The to-do list 614 is a tabular object corresponding to the tab 620. The to-do list 614 includes a status for each action corresponding to the suggestions for the selected person requiring nursing care. Such statuses are created and updated by the information collected via the input button 613. The to-do list 614 indicates the status of each action in association with the action, as illustrated in FIG. 10. With such a user interface 600, the user can easily grasp the status pertaining to the action corresponding to the suggestion. In particular, the user can easily grasp information such as which action is stalled, the time at which the action started to be stalled, who is in charge of the stalled action, and the reason why the action is stalled, thereby making care management more efficient.

FIG. 11 illustrates the user interface 600 when the tab 620 is selected by clicking or the like. As described above, when the tab 620 is selected by clicking or the like, information pertaining to completed processes is displayed. The information pertaining to the completed processes is information on the persons requiring nursing care who are targeted for process management and includes information such as information on subjects for whom all actions have been completed. In other words, the information pertaining to the completed processes includes information on completed actions for the persons requiring nursing care who are targeted for process management. The user interface 600 illustrated in FIG. 11 includes a search menu 621, a search result list 622, and a list of completed actions 623. The search menu 621 is an object to search for predetermined information from information pertaining to the completed processes. The search menu 621 in FIG. 11 provides a function to search for applicable information and display the information in the search result list 622 by specifying at least one of the conditions of the room number and the name of a person requiring nursing care. The search result list 622 is an object that lists information on persons requiring nursing care who correspond to the conditions specified by the search menu 621. When the information on a person requiring nursing care displayed in the search result list 622 is selected by clicking or the like, information on the completed actions corresponding to the selected information is displayed in the list of completed actions 623. The list of completed actions 623 is an object that lists information on completed actions for the person requiring nursing care selected in the search result list 622. With such a user interface 600, the user can easily grasp the completion performance of the status pertaining to the actions corresponding to the suggestions.

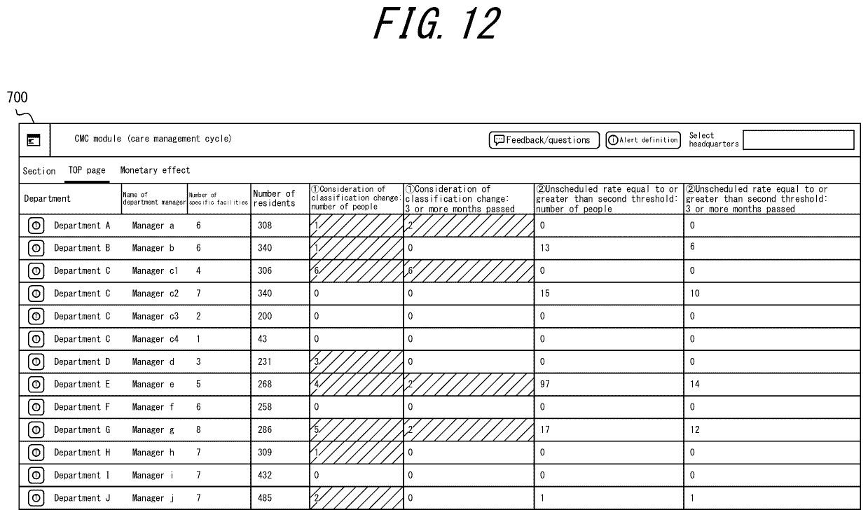

FIG. 12 is a diagram illustrating an example of a user interface in which aggregate information on the status for the aforementioned actions is displayed by the output interface 15 of the terminal apparatus 10. Such aggregate information is information for managers of a plurality of residential facilities (hereinafter also referred to as area managers), executives at the head office, and the like. The user interface 700 illustrated in FIG. 12 includes aggregate information that aggregates the information illustrated in FIGS. 10 and 11. Specifically, the aggregate information includes information on the number of persons requiring nursing care for whom a consideration of classification change was suggested, the number of persons requiring nursing care for whom three months or more have passed since the consideration of classification change was suggested, the number of persons requiring nursing care for whom a predetermined suggestion (such as warning about the unscheduled rate) was made, and the number of persons requiring nursing care for whom three months or more have passed since the predetermined suggestion was made. As illustrated in the user interface 700 in FIG. 12, the aggregate information is displayed as a tabular object. Here, cells that exceed a predetermined number of persons in the aggregate information on the consideration of classification change may be highlighted. The targets of aggregation in the aggregate information are only those persons requiring nursing care for whom the status of the action to be performed has not yet been inputted among the persons requiring nursing care who are targeted for process management. In other words, persons requiring nursing care for whom the status of the action to be performed has already been inputted are excluded from aggregation. For example, a person requiring nursing care whose status has already been entered as the result of a determination not to change the classification is excluded from the count in the corresponding aggregate information. By the target of the aggregate information thus being limited to persons requiring nursing care for whom the status of the action to be performed has not yet been inputted, it is possible to display aggregate results for only those persons requiring nursing care for whom action is required.

In the list of aggregate information in FIG. 12, specific information (such as the department in the first column) may be selectable by clicking or the like. Detailed information on the selected information may also be provided through another user interface. For example, when a department is selected, a list of facilities within that department may be displayed. In the list of facilities within the department, aggregate information for each facility may also be displayed. With this approach, analysis and countermeasures focused on individual facilities can be easily performed, such as which facility in a certain department is holding up actions that should be taken. Furthermore, specific information in the list of facilities can be selectable by clicking or the like. Detailed information on the selected information (such as information on the list of residents in the facility) may also be provided through another user interface. In addition, the user interface in FIG. 12 may be able to calculate and display the monetary effect and the like in the case of changing a classification. In this way, the care management cycle can be made more efficient by using the aggregate information in FIG. 12 as a starting point to facilitate access to more broken-down details, information on monetary effects, and the like. This information can also be used for monitoring purposes by area managers and by executives at the head office.

In the present embodiment, an example in which the nursing care database 220 is stored in the memory 22 of the information processing apparatus 20 has been illustrated, but this configuration is not limiting. For example, the nursing care database 220 may be stored on an external device such as a cloud server. In this case, the information processing apparatus 20 acquires the necessary information from the external device at steps S100 and S200 via the communication interface 23. Alternatively, part of the nursing care database 220 may be stored on an external device such as a cloud server. In this case as well, the information processing apparatus 20 acquires the necessary information from the external device at steps S100 and S200 via the communication interface 23, in addition to referring to the memory 22.

A case in which the information on the level of nursing care includes the first reference time and the second reference time has been described in the present embodiment, but this example is not limiting. For example, the information on the level of nursing care may include only one of the first reference time or the second reference time. In the present embodiment, an example in which the first reference time is a reference assistance time corresponding to a level of nursing care that is one level higher than the predetermined level of nursing care has been illustrated, but this configuration is not limiting. The first reference time may, for example, be a reference assistance time corresponding to a level of nursing care that is two or more levels higher than the predetermined level of nursing care. Alternatively, the first reference time may be any assistance time between a reference assistance time corresponding to a predetermined level of nursing care (second reference time) and a reference assistance time corresponding to a level of nursing care that is one level higher than the predetermined level of nursing care.

Although unscheduled care was defined as including toilet use care provided on an emergency basis, unscheduled care is not limited to this example. For example, unscheduled care includes emergency assistance in response to a nurse call. Unscheduled care also includes assistance with incontinence when toilet use care is not provided in time.

The suggestions in the present embodiment are not limited to the aforementioned examples. For example, the suggestions may include any warnings based on the implementation rate, the swing and miss rate, and the declination rate. For example, a case in which defecation has not occurred for a long period of time affects the implementation rate, the swing and miss rate, the declination rate, and the like are affected. The information processing apparatus 20 may present a suggestion indicating that such a case has occurred based on the implementation rate, the swing and miss rate, the declination rate, and the like. In other words, based on at least one of the implementation rate, the swing and miss rate, and the declination rate, an alert indicating that an action such as toilet use has not been performed for a certain period of time may be presented as a suggestion.

In the present embodiment, suggestions for the person requiring nursing care are presented based on the information on the level of nursing care, the schedule, and the performance information on care, but this configuration is not limiting. For example, suggestions pertaining to a person requiring nursing care may be presented based on ADL information in an assessment pertaining to the person requiring nursing care, or service plan information pertaining to various types of nursing care services and performance information on care. For example, in a case in which ADL information and performance (content/frequency) deviate from a predetermined reference, an alert may be presented as a suggestion. For example, an alert may be presented in a case in which a person is rated in the ADL information as independent in terms of toilet use, but toilet assistance has been performed a predetermined number of times or more. In a case in which the service plan information (Table 2/Table 3) and performance (content/frequency) deviate from a predetermined reference, an alert may be presented as a suggestion. For example, in a case in which Table 3 of the in-home service plan lists a certain number of instances of toilet assistance for each time slot of each day, an alert may be presented in a case in which toilet assistance is performed the predetermined number of times or more, toilet assistance occurs a certain number of times outside of the time slots listed in Table 3, incontinence occurs due to failure of toilet assistance, or a certain number or more of nurse calls are made for toilet assistance.

In the present embodiment, it is assumed that the schedule of care for a person requiring nursing care for whom a predetermined level of nursing care is set and the performance information on care for the person requiring nursing care are managed on an individual basis, but this configuration is not limiting. For example, the care schedule and performance information on care may be managed on a group basis for a plurality of persons requiring nursing care at a residential facility. In other words, in a residential facility that provides group assistance or the like, the schedule and performance information for the group may be managed on an individual basis or aggregated and managed for each predetermined group. In a case of management on a group basis, suggestions may be presented based on information on a predetermined level of nursing care, and based on the schedule and performance information on care for the corresponding group.

In the present embodiment, the level of nursing care can be evaluated at seven levels, i.e., nursing care level 1 to 5, and support level 1 to 2, but this configuration is not limiting. The level of nursing care can be any index indicating the level of nursing care or support needed in daily life and includes the levels defined in the Japanese nursing care level certification system, as well as similar indexes defined in other countries. Specifically, the level of nursing care may be information that can be classified into five levels (“grade 5”, “grade 4”, “grade 3”, “grade 2”, and “grade 1” in order starting from the mildest) as defined in the Korean system, for example. Alternatively, the level of nursing care may be a seven-level index (VV04 to VV10) as defined, for example, by Dutch long-term care insurance. The level of nursing care may also be a five-level index (nursing care levels 1?5) as defined, for example, by German public long-term care insurance.

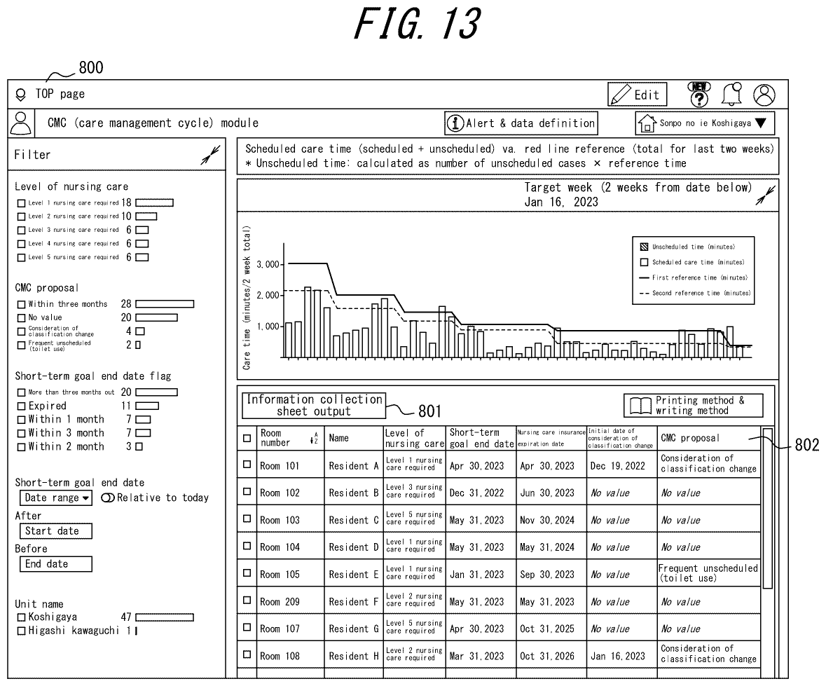

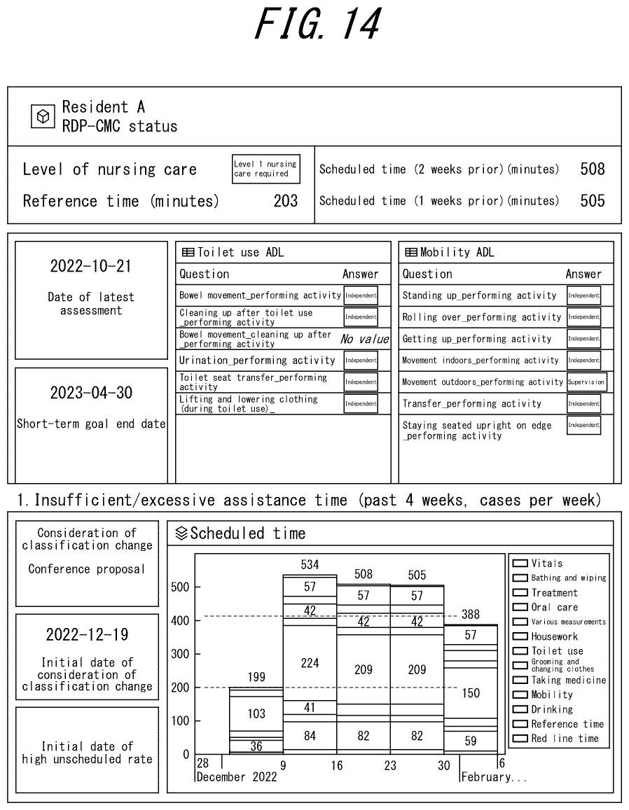

The controller 21 of the information processing apparatus 20 may output an information collection sheet pertaining to a person requiring nursing care via the output interface 15 of the terminal apparatus 10. The information collection sheet includes, for example, information pertaining to the level of nursing care, the schedule, and performance information on care. FIG. 13 illustrates an example of a user interface displayed by the output interface 15 of the terminal apparatus 10. FIG. 13 illustrates a user interface 800 provided to a user of a residential facility. As illustrated in FIG. 13, the user interface 400 includes an object 801 and a table 802. The object 801 is an object for outputting the information collection sheet. The table 802 illustrates various information including suggestions for each person requiring nursing care. For example, in the table 802, the resident targeted for output of the information collection sheet may be identified by a check box. When the object 801 is selected while a check box is selected, the information collection sheet is outputted. FIG. 14 is a diagram illustrating an example of an outputted information collection sheet. The information collection sheet illustrated in FIG. 14 includes a resident's name, level of nursing care, schedule, suggestions for the resident, ADL information, and the like. By including various types of information in this way, the information collection sheet enables the user easily to grasp the status of the person requiring nursing care.

Although the present disclosure is based on embodiments and drawings, it is to be noted that various changes and modifications will be apparent to those skilled in the art based on the present disclosure. Therefore, such changes and modifications are to be understood as included within the scope of the present disclosure. For example, the functions and the like included in the various units and steps may be reordered in any logically consistent way. Furthermore, a plurality of units and steps may be combined into one, or a single unit or step may be divided.

### REFERENCE SIGNS LIST

1 Care management system
10 Terminal apparatus
11 Controller
12 Memory
13 Communication interface
14 Input interface
15 Output interface
20 Information processing apparatus
21 Controller
22 Memory
23 Communication interface
30 Network
220 Nursing care database
400, 500, 600, 700, 800 Interface
410 to 415, 511 to 513, 801 Object
420 to 422 Graph
423 Bar graph
430, 520, 530, 802 Table
610, 620 Tab
611 List of ongoing processes
612 Selected person information
613 Input button
614 To-do list
621 Search menu
622 Search result list
623 List of completed actions

1. A care management support method to be executed by an information processing apparatus, the care management support method comprising:
acquiring a schedule of care for a person requiring nursing care for whom a predetermined level of nursing care is set;
acquiring performance information on care for the person requiring nursing care; and
presenting a suggestion for the person requiring nursing care based on information on the predetermined level of nursing care, the schedule, and the performance information on care.

2. The care management support method according to claim 1, wherein the information on the level of nursing care is a reference assistance time corresponding to a level of nursing care higher than the predetermined level of nursing care set for the person requiring nursing care.

3. The care management support method according to claim 1, wherein the performance information includes an implementation rate.

4. The care management support method according to claim 1, wherein the performance information includes an unscheduled rate.

5. The care management support method according to claim 1, wherein the performance information includes at least one of a swing and miss rate and a declination rate.

6. The care management support method according to claim 1, wherein the suggestion includes at least one of a change in classification of level of nursing care, a warning pertaining to an implementation rate, a warning pertaining to an unscheduled rate, and a warning pertaining to a swing and miss rate.

7. The care management support method according to claim 1, wherein the schedule includes a plan pertaining to predetermined care, the performance information includes performance pertaining to the predetermined care, and the suggestion includes a warning pertaining to the predetermined care.

8. The care management support method according to claim 7, wherein the predetermined care includes at least one of care pertaining to toilet use, mobility, drinking, taking medication, grooming and cleanliness, housework, and bathing.

9. The care management support method according to claim 1, further comprising displaying a list containing the suggestion corresponding to each person among a plurality of persons requiring nursing care.

10. The care management support method according to claim 1, further comprising presenting a status pertaining to an action corresponding to the suggestion.

11. A care management support method to be executed by an information processing apparatus, the care management support method comprising:
acquiring at least one of ADL information and service plan information pertaining to a person requiring nursing care for whom a predetermined level of nursing care is set;
acquiring performance information on care for the person requiring nursing care; and
presenting a suggestion for the person requiring nursing care based on the performance information on care and at least one of the ADL information and the service plan information.

12. The care management support method according to claim 1, further comprising outputting an information collection sheet.

13. An information processing apparatus comprising a controller, wherein
the controller is configured to
acquire a schedule of care for a person requiring nursing care for whom a predetermined level of nursing care is set;
acquire performance information on care for the person requiring nursing care; and
present a suggestion for the person requiring nursing care based on information on the predetermined level of nursing care, the schedule, and the performance information on care.

14. A system comprising the information processing apparatus according to claim 13 and a terminal apparatus, wherein
the terminal apparatus is configured to transmit performance information on nursing care for the person requiring nursing care to the information processing apparatus, and
the information processing apparatus is configured to acquire the performance information by receiving the performance information from the terminal apparatus.

15. (canceled)
